(19) United States
(12) Patent Application Publication
(10) Pub. No.: US 2017/0127992 A1
(43) Pub. Date: May 11, 2017

(54) FATIGUE-DEGREE MONITORING DEVICE, FATIGUE-DEGREE MONITORING SYSTEM, AND FATIGUE-DEGREE DETERMINING METHOD

(71) Applicant: Seiko Epson Corporation, Tokyo (JP)

(72) Inventor: Eizo Takahashi, Asahi-mura (JP)

(21) Appl. No.: 15/344,938

(22) Filed: Nov. 7, 2016

(30) Foreign Application Priority Data
Nov. 11, 2015 (JP) 2015-221004

(51) Int. Cl. A61B 5/16; A61B 5/024; A61B 5/11

(52) U.S. Cl. CPC A61B 5/161; A61B 5/1118; A61B 5/024

(57) ABSTRACT

A fatigue-degree monitoring device includes a first-fatigue-indicator calculating section configured to calculate a first indicator concerning fatigue of a user on the basis of operation from an operation section, a second-fatigue-indicator calculating section configured to calculate a second indicator concerning the fatigue of the user on the basis of biological information of the user, and a fatigue determining section configured to determine a fatigue degree of the user on the basis of the first indicator and the second indicator.

### CROSS-REFERENCES TO RELATED APPLICATIONS

This application claims priority to Japanese Patent Application No. 2015-221004, filed Nov. 11, 2015, the entirety of which is herein incorporated by reference.

### BACKGROUND

1. Technical Field

The present invention relates to a fatigue-degree monitoring device, a fatigue-degree monitoring system, and a fatigue-degree determining method.

2. Related Art

As one of measuring methods for mental fatigue, there has been known a flicker test. The flicker test makes use of a phenomenon in which, in a state in which a light source is flashed at high speed, flashing of light, that is, flickering (hereinafter referred to as flicker as well) cannot be recognized but, when speed, that is, a frequency of the flashing is reduced, the flicker starts to be recognized from a certain frequency. It is known that the frequency at which the recognition of the flicker starts is set as a threshold of the flicker recognition and the threshold changes according to mental fatigue. That is, according to fatigue, the threshold of the flicker recognition decreases, flashing at a high frequency cannot be recognized, and only flashing at a frequency lower than a frequency at healthy time can be recognized.

There has been proposed various methods and systems for measuring the threshold of the flicker recognition and measuring a fatigue degree using such a flicker test. For example, JP-A-2010-063641 (Patent Literature 1) discloses a method of causing, using a portable terminal apparatus, a user to notify recognition of a flicker through operation by the user and comparing frequency at the time when the user notifies the flicker and frequencies measured at healthy time and unhealthy time to thereby measure a fatigue degree of the user.

However, the recognition of the flicker is subjective operation based on visual recognition of the user. Therefore, when illumination or the like around the user affects the visual recognition of the flicker or the user arbitrarily performs the operation, an error occurs in the measurement of the threshold of the flicker recognition. The fatigue degree of the user cannot be accurately measured.

### SUMMARY

An advantage of some aspects of the invention is to accurately measure a fatigue degree of a user.

The invention can be implemented as the following forms or application examples.

### Application Example 1

A fatigue-degree monitoring device according to this application example includes: a first-fatigue-indicator calculating section configured to calculate a first indicator concerning fatigue of a user on the basis of an operation signal from an operation section; a second-fatigue-indicator calculating section configured to calculate a second indicator concerning the fatigue of the user on the basis of biological information of the user; and a fatigue determining section configured to determine a fatigue degree of the user on the basis of the first indicator and the second indicator.

With this configuration, the fatigue degree of the user is determined on the basis of two indicators, that is, the first indicator calculated on the basis of the operation signal from the operation section and the second indicator calculated on the basis of the biological information of the user. Therefore, it is possible to suppress the influence of an error caused by determining the fatigue degree of the user only with the first indicator based on subjective operation of the user and accurately determine the fatigue degree of the user.

### Application Example 2

In the fatigue-degree monitoring device according to the application example, it is preferable that the fatigue-degree monitoring device further includes an activity-amount calculating section configured to calculate an activity amount of the user on the basis of body motion information of the user, and the fatigue determining section determines the fatigue degree of the user on the basis of the activity amount.

With this configuration, it is possible to more accurately determine the fatigue degree of the user by determining the fatigue degree on the basis of the activity amount based on body motion information of the user in addition to the first indicator and the second indicator.

### Application Example 3

In the fatigue-degree monitoring device according to the application example, it is preferable that the fatigue-degree monitoring device further includes a fatigue predicting section configured to predict a transition in the fatigue degree involved in elapse of time on the basis of the first indicator, the second indicator, and the fatigue degree.

With this configuration, the transition of the fatigue degree is predicted on the basis of the first indicator and the second indicator, which fluctuate with the elapse of time, in addition to the determined fatigue degree. Therefore, it is possible to accurately predict the transition of the fatigue degree.

### Application Example 4

In the fatigue-degree monitoring device according to the application example, it is preferable that, when determining that the predicted fatigue degree exceeds a predetermined reference, the fatigue predicting section outputs an alert signal.

With this configuration, when determining that the predicted fatigue degree exceeds the predetermined reference, the fatigue predicting section outputs the alert signal. Therefore, it is possible to warn a fatigue state exceeding the predetermined reference.

### Application Example 5

In the fatigue-degree monitoring device according to the application example, it is preferable that the fatigue-degree monitoring device further includes a display section configured to display the fatigue degree determined by the fatigue determining section.

With this configuration, since the fatigue degree is displayed on the display section, the user can visually recognize information concerning the fatigue degree.

### Application Example 6

In the fatigue-degree monitoring device according to the application example, it is preferable that the fatigue-degree monitoring device further includes: a light emitting section configured to emit light; and an operation section configured to receive operation, and the first-fatigue-indicator calculating section calculates the first indicator on the basis of the operation signal on the operation section responding to the light emission of the light emitting section.

With this configuration, when the user operates the operation section in response to the light emission of the light emitting section, the first-fatigue-indicator calculating section can calculate the first indicator.

### Application Example 7

In the fatigue-degree monitoring device according to the application example, it is preferable that the light emitting section emits light according to a light emission frequency, and the first-fatigue-indicator calculating section calculates the first indicator on the basis of the light emission frequency at the time when the operation section is operated.

With this configuration, the first-fatigue-indicator calculating section can calculate the first indicator on the basis of the light emission frequency at the time when the user operates the operation section.

### Application Example 8

In the fatigue-degree monitoring device according to the application example, it is preferable that the fatigue-degree monitoring device further includes: a pulse-rate calculating section configured to calculate a pulse rate of the user on the basis of the biological information; and a zone determining section configured to determine a zone in which the pulse rate is included, and the light emitting section emits light according to the zone determined by the zone determining section.

With this configuration, the light emitting section can notify the zone corresponding to the pulse rate of the user by emitting light.

### Application Example 9

In the fatigue-degree monitoring device according to the application example, it is preferable that the first-fatigue-indicator calculating section acquires present time and calculates the first indicator when the acquired present time reaches predetermined time.

With this configuration, it is possible to calculate the first indicator at determined time.

### Application Example 10

In the fatigue-degree monitoring device according to the application example, it is preferable that the fatigue determining section determines the fatigue degree of the user by correcting the first indicator on the basis of the second indicator.

### Application Example 11

In the fatigue-degree monitoring device according to the application example, it is preferable that the fatigue-degree monitoring device further includes a transmitting section configured to transmit information concerning the fatigue degree to an external apparatus.

With this configuration, it is possible to transmit the information concerning the fatigue degree from the transmitting section to the external apparatus.

### Application Example 12

A fatigue-degree monitoring system according to this application example includes: a fatigue-degree monitoring device; and an information processing device. The fatigue-degree monitoring device includes: a first-fatigue-indicator calculating section configured to calculate a first indicator concerning fatigue of a user on the basis of an operation signal from an operation section; a second-fatigue-indicator calculating section configured to calculate a second indicator concerning the fatigue of the user on the basis of biological information of the user; a fatigue determining section configured to determine a fatigue degree of the user on the basis of the first indicator and the second indicator; and a transmitting section configured to transmit fatigue degree information concerning the fatigue degree determined by the fatigue determining section. The information processing device includes: a receiving section configured to receive the fatigue degree information; and a display section configured to display a transition of the received fatigue degree information in time series.

With this configuration, the fatigue degree of the user is determined on the basis of two indicators, that is, the first indicator calculated on the basis of the operation signal from the operation section and the second indicator calculated on the basis of the biological information of the user. Therefore, it is possible to suppress the influence of an error caused by determining the fatigue degree of the user only with the first indicator based on subjective operation of the user and accurately determine the fatigue degree of the user.

### Application Example 13

A fatigue-degree determining method according to this application example includes: calculating a first indicator concerning fatigue of a user on the basis of an operation signal; calculating a second indicator concerning the fatigue on the basis of biological information of the user; and determining a fatigue degree of the user on the basis of the first indicator and the second indicator.

With this method, the fatigue degree of the user is determined on the basis of two indicators, that is, the first indicator calculated on the basis of the operation signal from the operation section and the second indicator calculated on the basis of the biological information of the user. Therefore, it is possible to suppress the influence of an error caused by determining the fatigue degree of the user only with the first indicator based on subjective operation of the user and accurately determine the fatigue degree of the user.

### BRIEF DESCRIPTION OF THE DRAWINGS

The invention will be described with reference to the accompanying drawings, wherein like numbers reference like elements.

FIG. 1 is an exterior view showing the configuration of a fatigue-degree monitoring system according to an embodiment.

FIG. 2 is a block diagram showing the functional configuration of a device.

FIG. 3 is a side view of the device worn on an arm.

FIG. 4 is a front view of the device.

FIG. 5 is a diagram showing an image example displayed on a smartphone.

FIG. 6 is a diagram showing an image example displayed on the smartphone.

FIG. 7 is a diagram showing an image example displayed on the smartphone.

### DESCRIPTION OF EXEMPLARY EMBODIMENTS

An embodiment of the invention is explained below with reference to the drawings.

### Embodiment

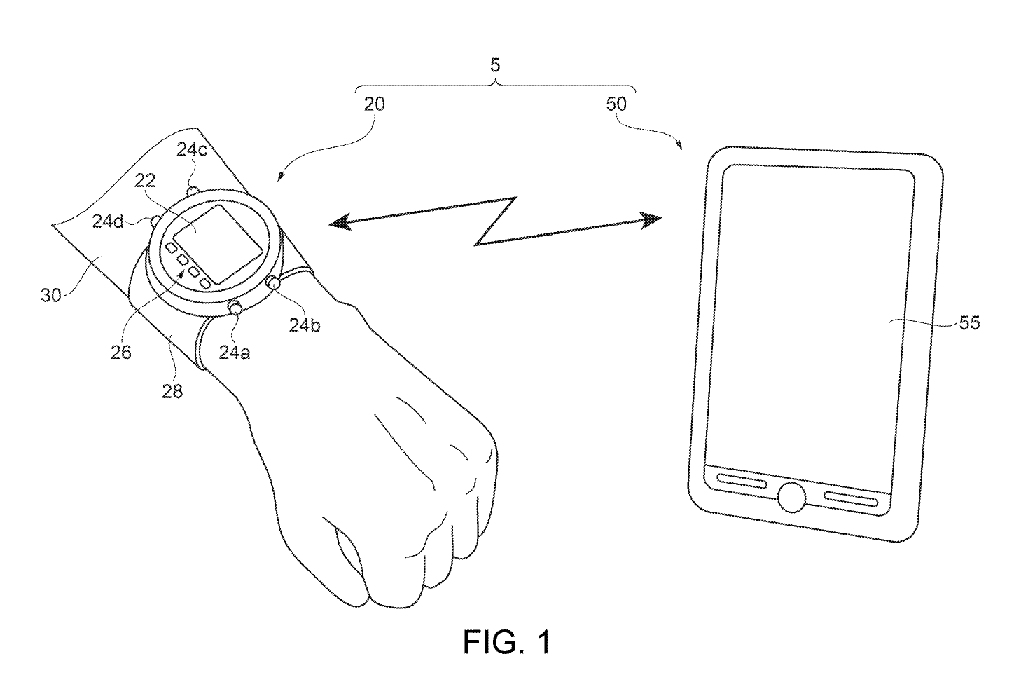

FIG. 1 is an exterior view showing the configuration of a fatigue-degree monitoring system 5 according to an embodiment. The fatigue-degree monitoring system 5 is a system that monitors a fatigue degree of a user. The fatigue-degree monitoring system 5 includes a device 20 functioning as a fatigue-degree monitoring device and a smartphone 50 functioning as an information processing device. The device 20 and the smartphone 50 are connected via communication.

The device 20 is assumed to be a wristwatch type. A display panel 22 and an indicator 26 are disposed on the surface of the device 20. Operation buttons (24a, 24b, 24c, and 24d) are disposed on a side surface of the device 20. The user can wear the device 20 on an arm 30 of the user using a belt 28 of the device 20. On the inside of the device 20, although not shown in the figure, hardware such as a CPU, a RAM, a ROM, a flash memory, and various electronic circuits is mounted.

The device 20 can measure a fatigue degree and the like of the user according to operation of the operation buttons (24a, 24b, 24c, and 24d) and can display measured information on the display panel 22 and the indicator 26.

The information measured by the device 20 is transmitted to the smartphone 50. The smartphone 50 includes a receiving section and can receive and store the transmitted information. Further, the smartphone 50 applies various kinds of information processing to the stored information according to operation of a touch panel 55 or the like by the user and displays a processing result on the touch panel 55. Note that the touch panel 55 is equivalent to a display section.

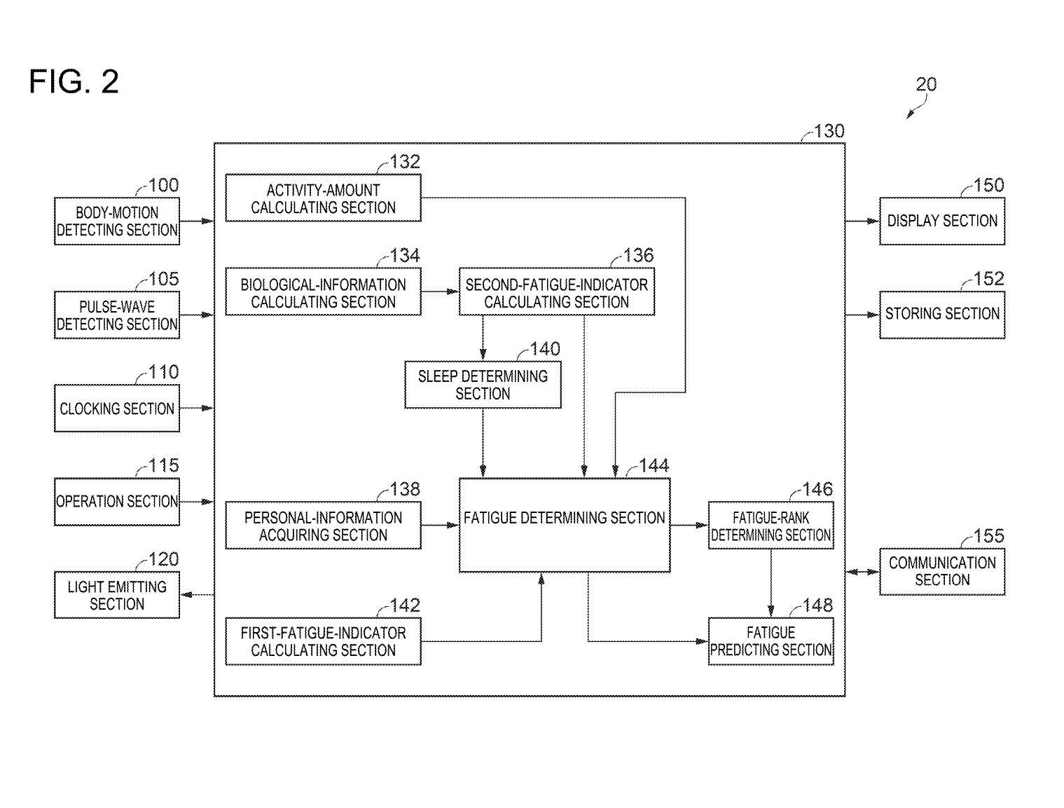

FIG. 2 is a block diagram showing the functional configuration of the device 20. The device 20 includes a body-motion detecting section 100, a pulse-wave detecting section 105, a clocking section 110, an operation section 115, a light emitting section 120, a control section 130, a display section 150, a storing section 152, and a communication section 155.

The body-motion detecting section 100 is configured by, for example, an element, a resistance value of which increases and decreases according to an external force. The body-motion detecting section 100 includes a motion sensor (an acceleration sensor) that detects acceleration information of three axes and a gyro sensor. The body-motion detecting section 100 detects a body motion of the user wearing the device 20 and outputs the body motion to the control section 130 as a body motion signal.

A pulse wave sensor 25 detects a pulse rate of the user wearing the device 20. The pulse-wave detecting section 105 outputs the pulse rate to the control section 130 as a pulse wave signal.

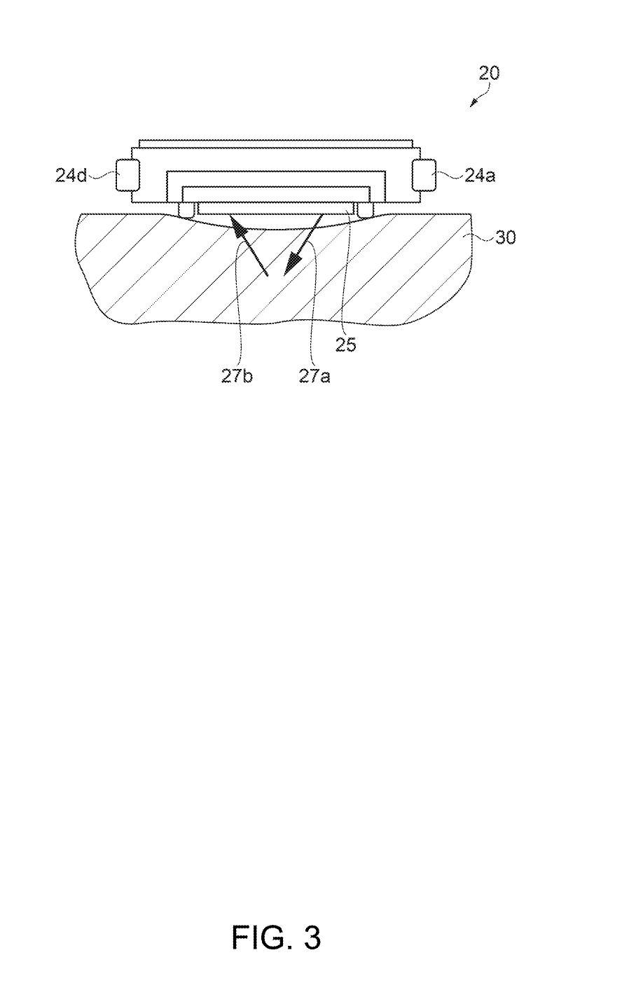

FIG. 3 is a side view of the device 20 worn on the arm 30. The pulse wave sensor 25 is disposed on a rear surface section of the device 20 to be opposed to the arm 30.

As the pulse wave sensor 25, for example, a photoelectric sensor including a light emitting element such as an LED and a light receiving element such as a photodiode can be assumed. In a state in which the device 20 is worn on the arm 30, the photoelectric sensor causes the light emitting element to radiate emitted light 27a toward an organism and detects a light amount change in receiving, with the light receiving element, reflected light 27b arriving through a blood vessel of the organism to output a pulse wave signal corresponding to a pulse wave.

Referring back to FIG. 2, the clocking section 110 acquires present time information by clocking time. As a clocking method, a so-called quartz system can be adopted. The clocking section 110 outputs the acquired time information to the control section 130.

The operation section 115 outputs an operation signal based on operation by the user to the control section 130. In this embodiment, the operation section 115 generates an operation signal according to operation of the operation buttons (24a, 24b, 24c, and 24d) and outputs the generated operation signal to the control section 130.

The light emitting section 120 flashes the indicator 26 according to an instruction signal from the control section 130. When a flicker value is calculated, the light emitting section 120 changes a light emission frequency of the indicator 26 and causes the user to visually recognize a contrast difference.

The display section 150 causes the display panel 22 to display information according to a display signal from the control section 130.

The storing section 152 stores data calculated by the control section 130. In this embodiment, the storing section 152 is assumed to be a flash memory.

The communication section 155 exchanges information with the smartphone 50 via radio communication on the basis of an instruction from the control section 130. In this embodiment, the communication section 155 is equivalent to a transmitting section that transmits information concerning a fatigue degree to an external apparatus. The communication section 155 is assumed to be a configuration including a transceiver corresponding to a short-range radio communication standard such as Bluetooth (registered trademark) (including BILE: Bluetooth Low Energy), Wi-Fi (registered trademark) (Wireless Fidelity), Zigbee (registered trademark), NFC (Near field communication), or ANT+ (registered trademark).

The control section 130 includes an activity-amount calculating section 132, a biological-information calculating section 134, a second-fatigue-indicator calculating section 136, a personal-information acquiring section 138, a sleep determining section 140, a first-fatigue-indicator calculating section 142, a fatigue determining section 144, a fatigue-rank determining section 146, and a fatigue predicting section 148.

These functional sections indicate functional components realized by cooperation of the hardware explained above and software stored in the ROM or the like. Therefore, hardware individually corresponding to the respective functional sections does not always need to be implemented. It is also possible to adopt a configuration in which one processor executes a computer program to realize functions of a plurality of functional sections. A part of functions realized by the software may be realized by the hardware. Alternatively, a part of functions realized by the hardware may be realized by the software.

The activity-amount calculating section 132 calculates an activity amount indicating an exercise state of the user on the basis of body motion information (a body motion signal) of the user output from the body-motion detecting section 100 and outputs the calculated activity amount to the fatigue determining section 144.

The biological-information calculating section 134 is equivalent to a pulse-rate calculating section. The biological-information calculating section 134 calculates biological information including a pulse rate of the user on the basis of a pulse-wave signal output from the pulse-wave detecting section 105 and outputs the calculated biological information to the second-fatigue-indicator calculating section 136.

Note that, as a method of calculating the activity amount from the body motion signal and a method of calculating the biological information from the pulse wave signal, for example, methods disclosed in JP-A-2013-208311 can be adopted.

The second-fatigue-indicator calculating section 136 analyzes the pulse rate included in the biological information sent from the biological-information calculating section 134, calculates, on the basis of a high-frequency component and a low-frequency component obtained by a frequency analysis of heart beat interval fluctuation, an autonomic nerve activity indicator (a second indicator) indicating a fatigue degree of the user, and outputs the calculated autonomic nerve activity indicator to the fatigue determining section 144 and the sleep determining section 140. Note that, as a method of calculating the fatigue degree from the frequency analysis of the heart beat interval fluctuation, for example, a method disclosed in JP-A-2015-109888 can be adopted.

The personal-information acquiring section 138 acquires personal information such as age and sex of the user and outputs the acquired personal information to the fatigue determining section 144. In this embodiment, the personal-information acquiring section 138 acquires a state of subjective fatigue felt by the user and outputs the state of the subjective fatigue to the fatigue determining section 144. Note that, in this embodiment, a form is assumed in which the personal information of the user is input by the user himself or herself.

The sleep determining section 140 determines a sleep state of the user on the basis of the autonomic nerve activity indicator sent from the second-fatigue-indicator calculating section 136 and outputs a determined result to the fatigue determining section 144.

The first-fatigue-indicator calculating section 142 calculates, on the basis of a central nervous system function and an autonomic nerve function, a first indicator indicating a fatigue degree of the user and outputs the calculated first indicator to the fatigue determining section 144.

In this embodiment, a well-known flicker value (CFF) is adopted as the first indicator. The first-fatigue-indicator calculating section 142 calculates the flicker value by causing the light emitting section 120 to flash the indicator 26 and causing the user, who visually recognizes the flashing, to operate the operation section 115 and respond.

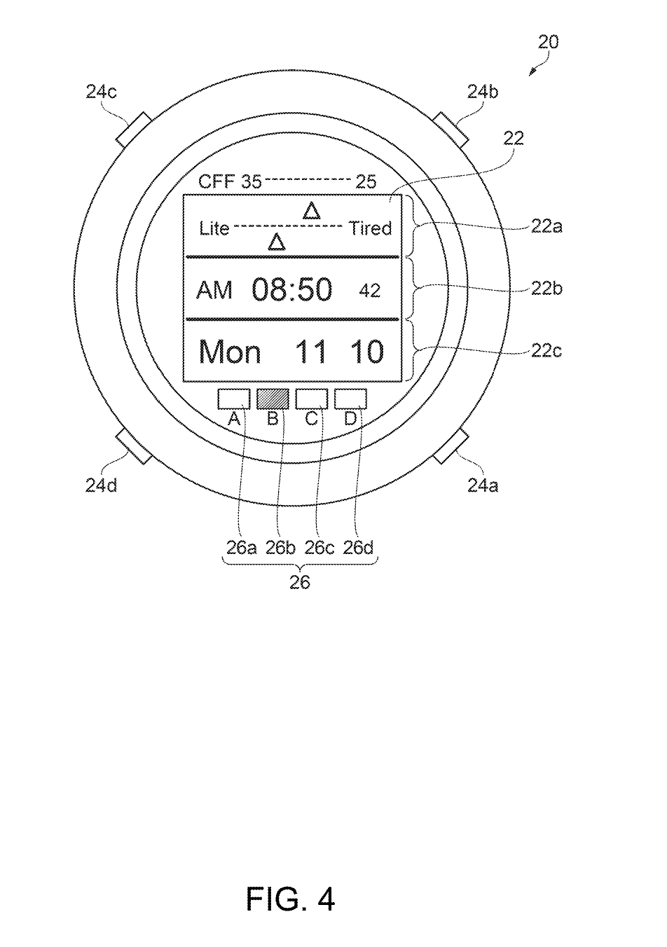

The indicator 26 is configured by LEDs 26. In this embodiment, the indicator 26 is configured by four LEDs (26a, 26b, 26c, and 26d) (FIG. 4) and disposed in a lower part of the display panel 22. The LEDs (26a, 26b, 26c, and 26d) individually flash according to an instruction signal sent from the first-fatigue-indicator calculating section 142.

When the light emitting section 120 flashes the indicator 26 at a light emission frequency and gradually reduces the light emission frequency and the flashing of the indicator 26 can be visually recognized, the first-fatigue-indicator calculating section 142 causes the user to depress one of the operation buttons (24a, 24b, 24c, and 24d). The first-fatigue-indicator calculating section 142 calculates a flicker value on the basis of a light emission frequency at the time when the operation button is depressed. In this case, by causing the user to depress the operation button 24 corresponding to a flashing part, it is possible to eliminate erroneous operation by the user and improve calculation accuracy of the flicker value.

Note that, when the LEDs (26a, 26b, 26c, and 26d) sequentially start flashing, the light emission frequency is gradually reduced, and flashing of a specific LED 26, for example, the LED 26b can be visually recognized, the indicator 26 may cause the user to depress the operation button 24b.

Note that, as a method of calculating the flicker value, the method described in Patent Literature 1 can be adopted.

Note that, in this embodiment, the device 20 is set to calculate the flicker value when the user depresses a predetermined operation button out of the operation buttons (24a, 24b, 24c, and 24d).

When the flicker value is not calculated, the biological-information calculating section 134 is set to calculate a pulse rate at a predetermined time interval, determine in which of zones classified in advance the calculated pulse rate is included (a zone determining section), and cause any one of the LEDs (26a, 26b, 26c, and 26d) corresponding to the relevant zone to emit light.

In this case, the activity-amount calculating section 132 may further calculate the number of steps, a consumed calorie, exercise strength, and the like of the user based on the calculated activity amount and display these kinds of information with the flashing of the LEDs (26a, 26b, 26c, and 26d).

The fatigue determining section 144 determines a mental fatigue degree of the user on the basis of the flicker value calculated by the first-fatigue-indicator calculating section 142 and the autonomic nerve activity indicator calculated by the second-fatigue-indicator calculating section 136. In this embodiment, the fatigue determining section 144 sets the calculated flicker value as a reference. That is, the fatigue determining section 144 determines a fatigue degree of the user by correcting the flicker value on the basis of the autonomic nerve activity indicator. However, not only this, but it is also possible to assume a form in which the autonomic nerve activity indicator is set as a reference and is corrected on the basis of the flicker value.

Further, when determining the fatigue degree, the fatigue determining section 144 may adopt at least one of the activity amount calculated by the activity-amount calculating section 132, the determination result determined by the sleep determining section 140, and attribute information of the user acquired by the personal-information acquiring section 138 and correct the determined fatigue degree. For example, when the sleep determining section 140 determines that the user had a deep sleep, the fatigue determining section 144 may determine that the user has recovered from fatigue through a high-quality sleep and reduce the determined fatigue degree. The sleep determining section 140 may correct the determined fatigue degree on the basis of the age, the sex, and the like of the user acquired by the personal-information acquiring section 138.

The fatigue determining section 144 outputs the determined fatigue degree to the fatigue-rank determining section 146. The fatigue determining section 144 outputs information concerning the autonomic nerve activity indicator and the activity amount used for the determination of the fatigue degree to the fatigue predicting section 148.

The fatigue-rank determining section 146 determines a fatigue rank of the user on the basis of a determination result sent from the fatigue determining section 144. For example, as the fatigue rank, three levels of “normal”, “slightly tired”, and “tired” are assumed. The fatigue-rank determining section 146 sends information concerning the determined fatigue rank to the fatigue predicting section 148.

Further, the fatigue-rank determining section 146 may send the information concerning the fatigue rank to the display section 150 and cause the display section 150 to display the information concerning the fatigue rank. The fatigue-rank determining section 146 may transmit the information concerning the fatigue rank from the communication section 155 to the smartphone 50.

The fatigue predicting section 148 predicts a future transition concerning fatigue of the user on the basis of information concerning a present fatigue rank sent from the fatigue-rank determining section 146 and changes in the autonomic nerve activity indicator and the activity amount involved in the elapse of time after the determination of the fatigue degree. When determining that the fatigue degree of the user exceeds a predetermined threshold, the fatigue predicting section 148 outputs an alert signal and notifies, via the display panel 22 and the indicator 26, the user of a message for urging the user to take a rest.

FIG. 4 is a front view of the device 20. The display section 150 can divide the display panel 22 into three regions of an upper part 22a, a middle part 22b, and a lower part 22c and display various kinds of information in the regions. Note that, in FIG. 4, the display panel 22 displays a normal screen indicating a state of waiting for an instruction from the user.

In FIG. 4, in the upper part 22a, the flicker value and the fatigue rank are indicated by triangle marks. The triangle mark of the flicker value indicates a position corresponding to a range (35 Hz to 25 Hz) of the flicker value printed in an upper part. The position of the triangle mark is updated every time when the flicker value is calculated. A state of time set in advance (e.g., wakeup time) can also be retained. In this case, another triangle mark may be further displayed in the upper part 22a.

The triangle mark of the fatigue degree indicates any one of states “Light” to “Tired” according to the fatigue rank determined by the fatigue-rank determining section 146.

In general, fatigue feeling of the user gradually increases as the user wakes up and acts. Therefore, the triangle mark of the fatigue rank gradually moves in the direction of “Tired” from the wakeup.

The present time is displayed in the middle part 22b. The day of the week and month and day are displayed in the lower part 22c.

Note that a display mode of the lower part 22c is changed according to operation of the operation buttons (24a, 24b, 24c, and 24d).

For example, when the operation button 24c is depressed in a state shown in FIG. 4, the device 20 transitions to a flicker measurement mode and displays “Flicker mode” in the lower part 22c. When the operation button 24d is pressed in this state, the device 20 starts measurement of the flicker value and displays “Measuring now” in the lower part 22c.

Note that the transition to the flicker measurement mode is not limited to the depression of the operation button 24c by the user. For example, when, for example, time for the user to go to work in the morning is set in an alarm set mode explained below, when the set time comes, the device 20 can transition to the flicker measurement mode.

When the device 20 transitions to the flicker measurement mode and “Measuring now” is displayed in the lower part 22c, the LEDs (26a, 26b, 26c, and 26d) start flashing and cause the user to depress the operation button 24b. As a result, the first-fatigue-indicator calculating section 142 calculates the flicker value on the basis of a light emission frequency of the LED corresponding to the pressed operation button 24b. The display section 150 updates the position of the triangle mark of the flicker value in the upper part 22a.

Before the operation button 24b is depressed, the second-fatigue-indicator calculating section 136 acquires a state of an autonomic nerve on the basis of the pulse rate calculated by the biological-information calculating section 134 and stores the state of the autonomic nerve in the storing section 152. Note that, since the pulse rate calculated before the operation button 24b is depressed is adopted, it is possible to exclude the influence of fluctuation in a pulse rate of the user that occurs because of tension in depressing the operation button 24b.

After calculating the flicker value in the flicker measurement mode, the device 20 may inquire the user of a fatigue state and cause the user to input a fatigue state due to subjectivity of the user. For example, the device 20 may display “Are you tired?” in the lower part 22c and cause the user to depress one of the operation buttons (24a, 24b, 24c, and 24d) according to a rank of fatigue. The fatigue determining section 144 may correct the determined fatigue degree taking into account the fatigue state by subjectivity of the user.

Note that, when the operation button 24c is depressed in a state in which the device 20 transitions to the flicker measurement mode, the device 20 transitions to a heart rate measurement mode and displays “Heart rate mode” in the lower part 22c. When the operation button 24d is depressed in this state, the device 20 starts measurement of a heart rate and displays the measured heart rate in the lower part 22c.

When the operation button 24c is depressed in a state in which the device 20 transitions to the heart rate measurement mode, the device 20 transitions to an activity amount measurement mode and displays “Activity mode” in the lower part 22c. When the operation button 24d is depressed in this state, the device 20 starts measurement of an activity amount and displays information, for example, a consumed calorie corresponding to the measured activity amount in the lower part 22c.

When the operation button 24c is depressed in the state in which the device 20 transitions to the activity amount measurement mode, the device 20 transitions to a setting mode.

When the operation button 24d is depressed in a state in which the device 20 transitions to the setting mode, the device 20 sequentially displays a submenu of the setting mode. In this embodiment, in the submenu, one of “Time set mode”, “Alarm set mode”, and “Personal Inf mode” (personal information mode) is sequentially selected every time the operation button 24c is depressed.

In the time set mode, the present time and month and day can be set.

In the personal information mode, personal information including age, sex, height, and weight of the user can be set.

In a state in which the device 20 transitions to the alarm set mode, every time the operation button 24c is depressed, the device 20 sequentially selects one of time setting for urging the user to perform a flicker test, time setting for urging the user to take a rest, and setting of a sound type and a vibration type informed by an alarm.

Note that the alarm for urging the user to take a rest operates when set time comes. In addition, the alarm may operate when a set threshold is exceeded, for example, when a state in which the activity amount increases exceeds a predetermined time or when duration of a sympathetic nerve activity in the autonomic nerve exceeds a predetermined time. Note that the predetermined time may be corrected on the basis of the flicker value, age, a sleeping state in the previous day, or the like.

The alarm may operate in a degree in which a fatigue degree estimated from a change in the activity amount or a fatigue degree estimated from the autonomic nerve state does not exceed a predetermined reference value. Note that the reference value of the fatigue degree may be corrected on the basis of the flicker value, age, a sleeping state in the previous day, or the like.

Note that the device 20 is set to return to a normal screen when the operation button 24c is depressed in the state in which the device 20 transitions to the setting mode.

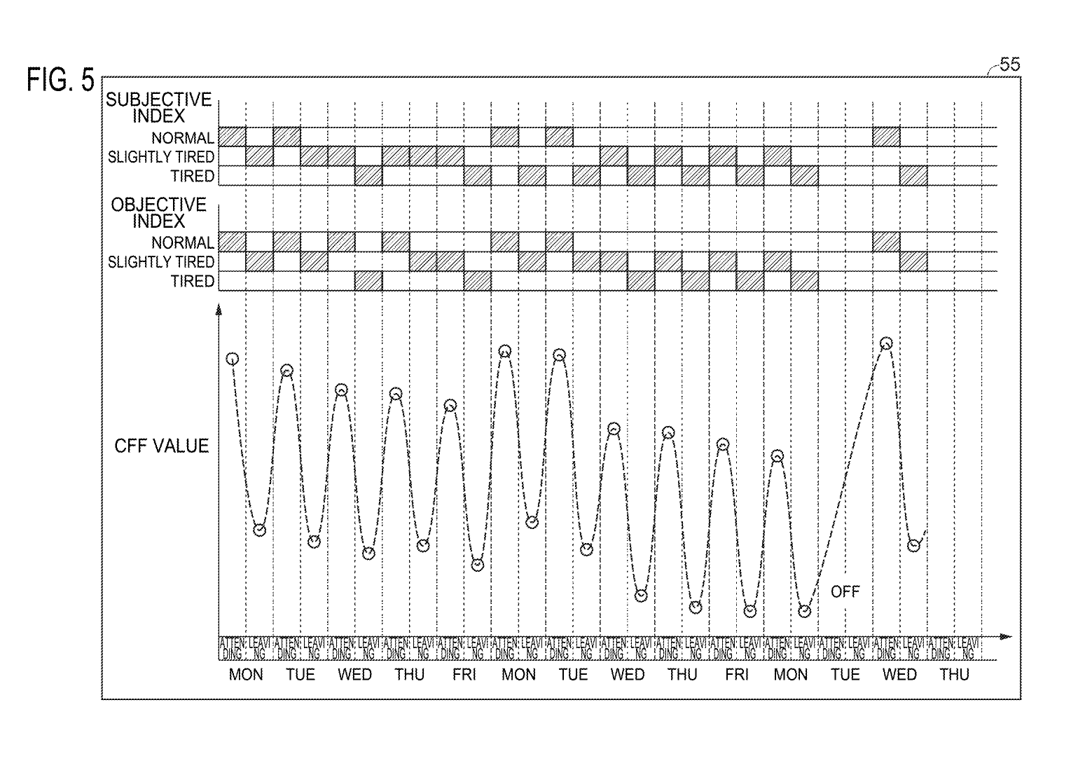

FIGS. 5 to 7 show examples of results of processing in which the smartphone 50 receives information transmitted from the device 20, applies information processing to the information, and displays everyday changes in time series on the touch panel 55.

FIG. 5 shows changes in a flicker value, a measured fatigue degree, and a fatigue degree input by the user tested when the user arrives at the office and when the user leaves the office in a predetermined number of days.

The fatigue degree determined by the device 20 is indicated by three levels of “normal”, “slightly tired”, and “tired” as objective indicators. The fatigue degree input by the user is indicated by three levels of “normal”, “slightly tired”, and “tired” as subjective indicators. The flicker value is represented as “CFF value” and fluctuation in the flicker value is indicated by a graph.

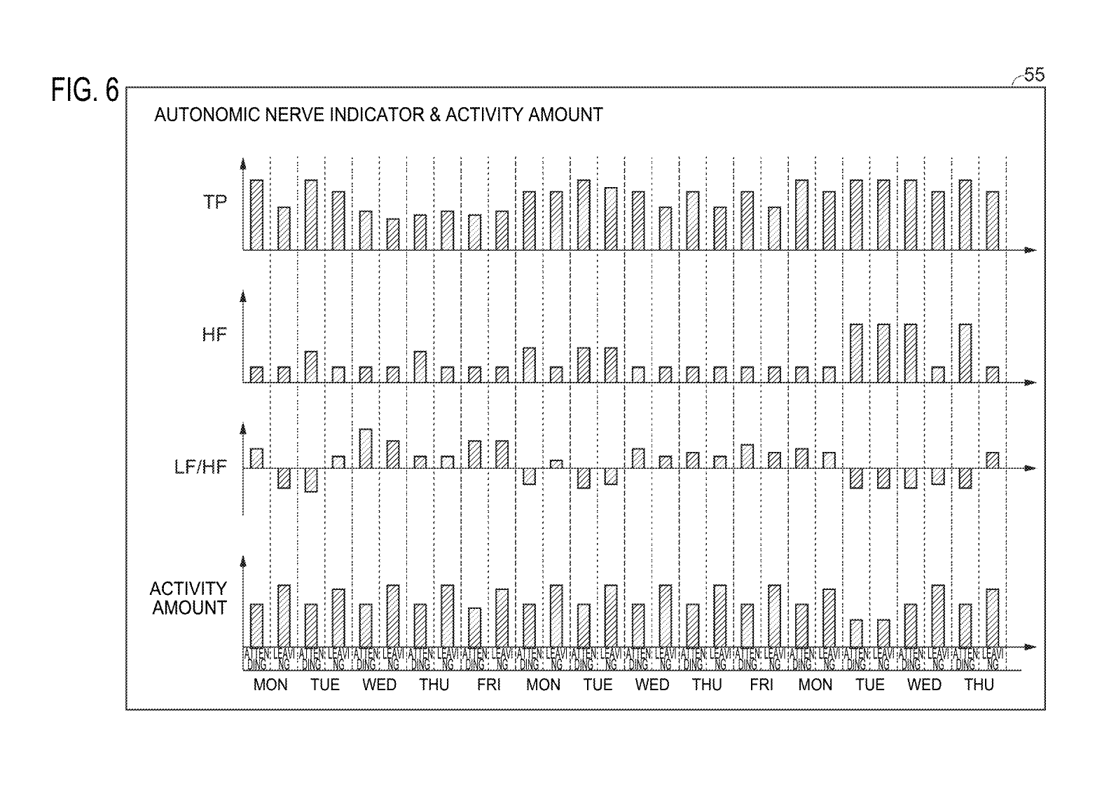

FIG. 6 shows a change in a predetermined number of times of an activity amount and an autonomic nerve activity state. In FIG. 6, a frequency analysis of heart rate fluctuation is performed on the basis of the autonomic nerve activity indicator. A high-frequency component (HF) of a power spectrum, a low-frequency component (LF) of the power spectrum, a ratio (LF/HF) of the high-frequency component (HF) and the low-frequency component (LF), and a change in total power (TP) in an entire region of the power spectrum are displayed. It is possible to analyze depth and quality of sleep from these kinds of information.

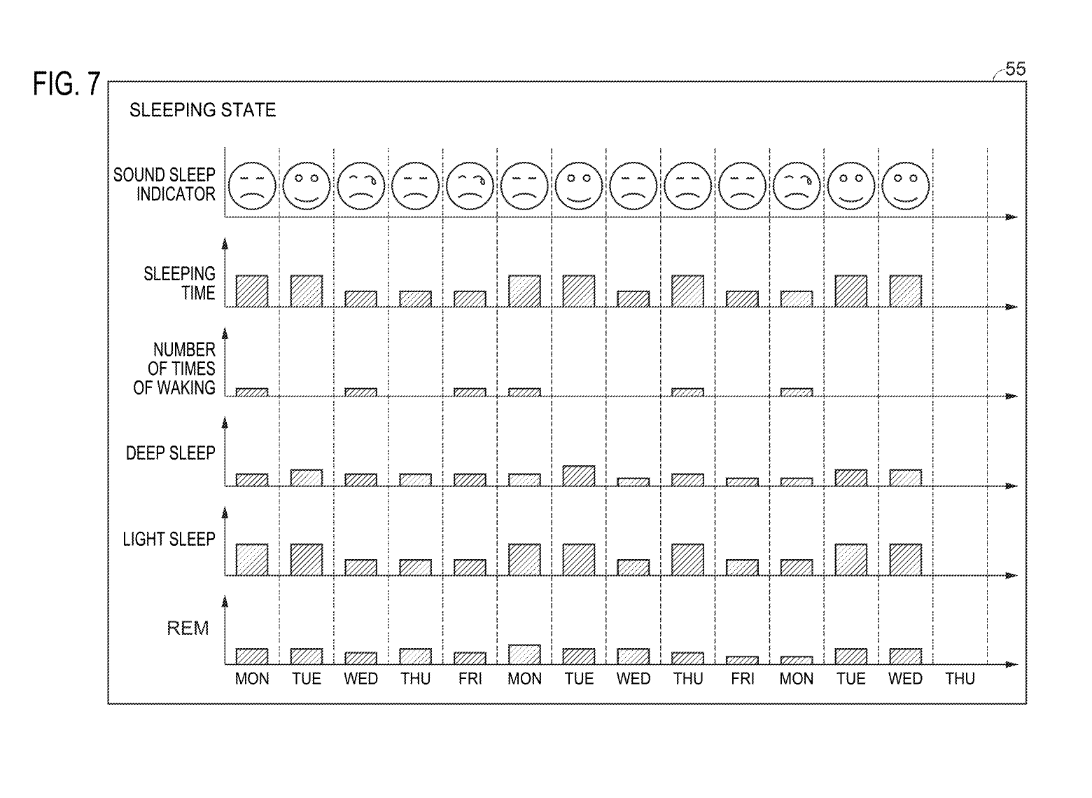

In FIG. 7, a change in a sleeping state is estimated from a change in biological information in a predetermined number of days. In the sleeping state, sound sleep indicators are determined on the basis of a sleeping time, the number of times of waking, deep sleep, light sleep, and REM sleep. Determination results are indicated by face marks of three levels as the sound sleep indicators.

Note that the smartphone 50 may transmit a processing result of the user to an external server apparatus (not shown in the figure) via the Internet or the like and receive and display analysis results of analysis processing of processing results of a plurality of users by the server apparatus. For example, it is also possible to assume a form in which a fatigue degree, an activity amount, an autonomic nerve activity state, and a sleeping state of the user are compared with members of a group to which the user belongs and a result of the comparison is displayed on the touch panel 55.

As explained above, with the fatigue-degree monitoring system 5 according to this embodiment, effects explained below can be obtained.

(1) The fatigue determining section 144 determines a fatigue degree of the user on the basis of two indicators, that is, the flicker value calculated by the first-fatigue-indicator calculating section 142 on the basis of the response operation by the user and the autonomic nerve activity indicator calculated by the second-fatigue-indicator calculating section 136 on the basis of the biological information of the user. Therefore, it is possible to eliminate erroneous determination caused by arbitrary operation by the user when the fatigue degree of the user is determined with only the flicker value. It is possible to determine the fatigue degree with high reliability.

(2) Further, in addition to the two indicators, the fatigue determining section 144 may adopt at least one of the activity amount calculated by the activity-amount calculating section 132, the determination result determined by the sleep determining section 140, and the attribute information of the user acquired by the personal-information acquiring section 138 and corrects the determined fatigue degree. Therefore, it is possible to further improve the reliability of the fatigue degree.

(3) The fatigue predicting section 148 can predict, from the present fatigue state, a change in a fatigue state involved in the elapse of time. Therefore, for example, in addition to determination of a fatigue degree limited to a determined place or determined time such as before a work start and after a work end, it is possible to detect a change in a fatigue degree during work and, when the change is about to exceed a reference, display a warning message. Therefore, it is possible to prevent a decrease in efficiency of work and a work mistake due to fatigue.

The change in the fatigue state involved in the elapse of time can be visually recognized by the smartphone 50. Therefore, it is possible to easily visually recognize the quality of sleep and a transition of a fatigue degree in a predetermined number of times. Therefore, it is easy to perform physical condition monitoring such as monitoring of fatigue, sleep, and the like of the user in a plurality of days.

Note that, in this embodiment, a configuration is assumed in which the fatigue-degree monitoring system 5 is divided into the device 20 and the smartphone 50. However, the invention is not limited to this. For example, a form can also be assumed in which the functions of the fatigue-degree monitoring system 5 are realized by the device 20.

Note that the device 20 is not limited to the wristwatch type and may be a finger ring type or a pendant type.

The information processing device is not limited to a high-function cellular phone such as the smartphone 50. A multifunction portable terminal such as a tablet terminal can also be assumed.

What is claimed is:

1. A fatigue-degree monitoring device comprising:
a first-fatigue-indicator calculating section configured to calculate a first indicator concerning fatigue of a user on the basis of an operation signal from an operation section;
a second-fatigue-indicator calculating section configured to calculate a second indicator concerning the fatigue of the user on the basis of biological information of the user; and
a fatigue determining section configured to determine a fatigue degree of the user on the basis of the first indicator and the second indicator.

2. The fatigue-degree monitoring device according to claim 1, further comprising an activity-amount calculating section configured to calculate an activity amount of the user on the basis of body motion information of the user, wherein
the fatigue determining section determines the fatigue degree of the user on the basis of the activity amount.

3. The fatigue-degree monitoring device according to claim 1, further comprising a fatigue predicting section configured to predict a transition in the fatigue degree involved in elapse of time on the basis of the first indicator, the second indicator, and the fatigue degree.

4. The fatigue-degree monitoring device according to claim 3, wherein, when determining that the predicted fatigue degree exceeds a predetermined reference, the fatigue predicting section outputs an alert signal.

5. The fatigue-degree monitoring device according to claim 1, further comprising a display section configured to display the fatigue degree determined by the fatigue determining section.

6. The fatigue-degree monitoring device according to claim 1, further comprising:
a light emitting section configured to emit light; and
an operation section configured to receive operation by the user, wherein
the first-fatigue-indicator calculating section calculates the first indicator on the basis of the operation signal from the operation section detected when the light emitting section is emitting light.

7. The fatigue-degree monitoring device according to claim 6, wherein
the light emitting section emits light at different light emission frequencies, and
the first-fatigue-indicator calculating section calculates the first indicator on the basis of the light emission frequency at a time when the operation section is operated.

8. The fatigue-degree monitoring device according to claim 6, further comprising:
a pulse-rate calculating section configured to calculate a pulse rate of the user on the basis of the biological information; and
a zone determining section configured to determine a zone in which the pulse rate is included, wherein
the light emitting section emits light at the light emission frequency according to the zone determined by the zone determining section.

9. The fatigue-degree monitoring device according to claim 1, wherein the first-fatigue-indicator calculating section acquires present time and calculates the first indicator when the acquired present time reaches predetermined time.

10. The fatigue-degree monitoring device according to claim 1, wherein the fatigue determining section determines the fatigue degree of the user by correcting the first indicator on the basis of the second indicator.

11. The fatigue-degree monitoring device according to claim 1, further comprising a transmitting section configured to transmit information concerning the fatigue degree to an external apparatus.

12. A fatigue-degree monitoring system comprising:
a fatigue-degree monitoring device; and
an information processing device, wherein
the fatigue-degree monitoring device including:
a first-fatigue-indicator calculating section configured to calculate a first indicator concerning fatigue of a user on the basis of an operation signal from an operation section;
a second-fatigue-indicator calculating section configured to calculate a second indicator concerning the fatigue of the user on the basis of biological information of the user;
a fatigue determining section configured to determine a fatigue degree of the user on the basis of the first indicator and the second indicator; and
a transmitting section configured to transmit fatigue degree information concerning the fatigue degree determined by the fatigue determining section, and
the information processing device includes:
a receiving section configured to receive the fatigue degree information; and
a display section configured to display a transition of the received fatigue degree information in time series.

13. A fatigue-degree determining method comprising:
calculating a first indicator concerning fatigue of a user on the basis of an operation signal;
calculating a second indicator concerning the fatigue on the basis of biological information of the user; and
determining a fatigue degree of the user on the basis of the first indicator and the second indicator.

14. The fatigue-degree determining method according to claim 13, further comprising:
calculating an activity amount of the user on the basis of body motion information of the user; and
determining the fatigue degree of the user on the basis of the activity amount.

15. The fatigue-degree determining method according to claim 13, further comprising predicting a transition in the fatigue degree involved in elapse of time on the basis of the first indicator, the second indicator, and the fatigue degree.

16. The fatigue-degree determining method according to claim 15, further comprising outputting an alert signal when determining that the predicted fatigue degree exceeds a predetermined reference.

17. The fatigue-degree determining method according to claim 13, further comprising:
causing a light emitting section to emit light; and
calculating the first indicator on the basis of the operation signal from the user detected when causing the light emitting section to emit light.

18. The fatigue-degree determining method according to claim 17, further comprising
emitting light at different light emission frequencies; and
calculating the first indicator on the basis of the light emission frequency at a time when the operation signal is detected.

19. The fatigue-degree determining method according to claim 17, further comprising:
calculating a pulse rate of the user on the basis of the biological information;
determining a zone in which the pulse rate is included; and
emitting light at the light emission frequency corresponding to the determined zone.

20. The fatigue-degree determining method according to claim 13, further comprising determining the fatigue degree of the user by correcting the first indicator on the basis of the second indicator.
